(19) United States
(12) Patent Application Publication
(10) Pub. No.: US 2006/0182704 A1
(43) Pub. Date: Aug. 17, 2006

(54) Disposable Whole Face Mask for Topical Delivery of Cosmetic, Nutritional or Medicinal Preparations and for use with Facial Massage, Acupressure and Aroma Therapies and the Method of Applying Topical Cosmetic, Nutritional or Medicinal Preparations thereby

(76) Inventor: Penelope Marie Gianelli, Orlando, FL (US)

Correspondence Address: PENELOPE M. GIANELLI, 1015 N. LAKE DAVIS DR., ORLANDO, FL, 32806, US

(21) Appl. No.: 10/906,339

(22) Filed: Feb. 15, 2005

(51) Int. Cl. A61K 8/64; A61K 8/97

(52) U.S. Cl. 424/70.14; 424/74

(57) ABSTRACT

The invention describes a novel invention of a disposable mask which covers the entire face and is pre-moistened or impregnated with a cosmetic, nutritional or medicinal solution, preferably one beneficial to the skin; as well as the novel invention of the method of applying cosmetic, nutritional or medicinal solutions to the face by such a disposable face mask; as well as the invention of and the use of such face mask impregnated with aromatic fragrances and/or imprinted with facial massage information and facial acupressure points. This invention is beneficial in the field of skin care products for ameliorating irritating skin conditions, improving the skin's visual appearance and biophysical function, providing aroma therapy and providing a smooth, moist surface, in some instances pre-printed with directions, for facial massage and acupressure stimulation.

### CROSS REFERENCE TO RELATED APPLICATIONS

Not Applicable

### STATEMENT REGARDING FEDERALLY SPONSORED RESEARCH OR DEVELOPMENT

Not Applicable

### BACKGROUND OF INVENTION

The human face is of supreme importance in daily life. We “face” the world literally—using our face to communicate with and make an impression on others. Our faces have some of the most delicate skin and active muscles of our entire body and yet, at the same time, are exposed to the physical elements and worked intensively 24 hours a day, as facial expressions continue even in sleep. Current face “treatments” generally involve applying creams or lotions to the skin surface where they are left to dry.

Environmental conditions, including radiation, temperature changes, lack of moisture, sun exposure, adverse environmental pollutants, household chemicals, smoking, wind exposure, as well as, chronological age, contribute to the aging and deterioration of skin cells and facial appearance which is considered cosmetically undesirable. These include the development of fine lines and wrinkles, loss of elasticity and firmness, increased sagging, loss of color evenness or tone, coarse surface texture and mottled pigmentation.

Acne, eczema and other inflammatory skin conditions affect millions of people and can mar the skin's appearance. Unsightly skin can substantially damage an individual's self-esteem.

Over 14 million people in the United States are estimated to have a skin condition called rosacea, a chronic cutaneous disorder affecting primarily the convexities of the central face, in which continued erythema (flushing) causes very dry, red skin. In advanced stages, persistent erythema (flushing) leads to the build up of layers of rough red plaques causing a bumpy appearance. Rosacea causes significant psychological, social and occupational problems if left untreated according to the National Rosacea Society.

Unfortunately, these, and many other, skin conditions do not have a cure and are chronic conditions that are covered up make-ups or temporarily ameliorated by ointments or creams.

Numerous skin conditions and disorders require the direct, topical delivery of cosmetic, nutritional or medicinal solutions. Certain minimum, continuous exposure times are required for various topical applications to be sufficiently absorbed by the skin to be effective to achieve an intended result or for a liquid to be fully absorbed through the epidermis and into the dermis or deeper levels of the skin. In other instances, the efficacy of cosmetic, nutritional and medicinal topical applications is improved by continued moist contact with the skin for a period of time. Topical applications that are put on the skin and dry in place, evaporate and lose effectiveness in the drying process, thereby ceasing to function. The present invention offers a gentle and relaxing method for the delivery of ingredients and for gradual, more complete, and more effective deep absorption into the skin by allowing the application to remain moist and active state longer and in contact with the skin.

Many cosmetic preparations utilize synthetic chemicals, dyes, fragrances and compounds which are irritating or cause allergic sensitivities, but are incorporated into products to produce a commercially acceptable or desirable fragrance, color or consistency for people to apply and leave on their skin. As a result, people, especially those with sensitive or irritated skin, are seeking more natural and soothing methods of skin care and treatment. The face mask offers an alternative method of applying preparations on the face without the need to leave the preparation remaining on the skin beyond the treatment time, thereby eliminating the need for additives which have the sole function of creating a preparation appropriate for application to the skin.

Relaxing facial treatments can be a refreshing respite from the hectic pace of modern life. Aroma therapies can have relaxing or invigorating effects. Soothing facial treatments, including facial massage or acupressure point massage and/or stimulation can further enhance the skin treatment process or be administered as stand alone treatments.

Salon facials are very expensive and require considerable time and effort, which is not always possible, going to and from a salon. Peel off masque preparations are messy, uncomfortable and stick to the user's hair, sometimes, pulling it out painfully upon removal of the masque preparation.

Accordingly, it is an object of the present invention to provide a facial mask pre-impregnated with a cosmetic composition to be placed over the entire face, designed as an alternative to peel off or wash off “masque” cosmetic preparations, for use as a facial cosmetic or treatment.

Another object of the present invention is to provide a facial mask pre-impregnated with a nutritional or medicinal composition to be applied to the face.

A further object of the present invention is to provide a facial mask pre-moistened with an aqueous fluid that provides an aromatic, soothing and relaxing experience to the user as tool for aroma therapy and relaxation.

Still a further object of the present invention is to provide a facial mask printed on one side with massage directions and acupressure point locations and also pre-moistened with an aqueous fluid that provides a smooth, moist surface over the face enabling and facilitating facial massage and acupressure point stimulation by the user or a third party.

### BRIEF DESCRIPTION OF INVENTION

The invention describes a novel invention of a disposable mask which covers the entire face and is pre-moistened or impregnated with a cosmetic, nutritional or medicinal solution, preferably one beneficial to the skin; as well as the novel invention of the method of applying cosmetic, nutritional or medicinal solutions to the face by such a disposable face mask; as well as the invention and use of such face mask impregnated with aromatic fragrances and/or imprinted with facial massage information and facial acupressure points. This invention is beneficial in the field of skin care products for ameliorating irritating skin conditions, improving the skin's visual appearance and biophysical function, providing topical delivery of nutritional or medical treatments, providing aroma therapy and providing a smooth, moist surface, in some instances pre-printed with directions, for facial massage and acupressure stimulation.

### DESCRIPTION OF DRAWINGS

Further features, objects and advantages of the present invention can be understood from the drawings:

FIG. 1 is flat view of the face mask material, in an oval shape approximately 8 inches high by 9 inches wide, showing the eye hole openings, nose hole opening flap, mouth hole and radial slit openings.

FIG. 2 is a flat view of the face mask material as in FIG. 1 with the additional feature of printed massage directions and acupressure points; the printing shown is suggestive only of the different directions and points that could be printed depending upon the type of massage or purpose of acupressure treatment for which the face mask is intended.

FIG. 3 is a perspective view of a foil pouch, torn at the top to reveal the folded face mask and the dripping of the solution which is in the face mask material and in the pouch.

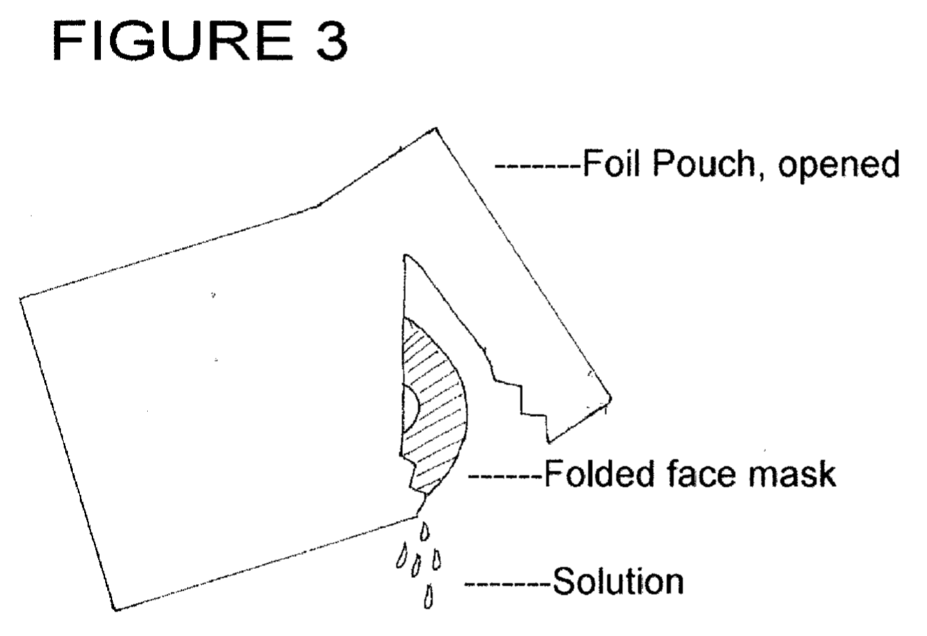

FIG. 4 is a woman's face with the face mask in position.

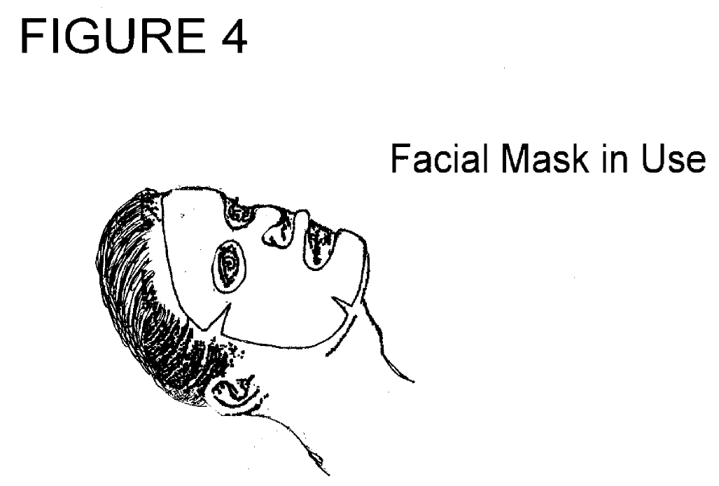

### DETAILED DESCRIPTION OF INVENTION

The features, objects and advantages of the invention may be understood, made, and used by reference to the drawings, claims, and the descriptions in this application. Further features of the invention are apparent from the entirety of the specification or may be learned through the practice of the invention.

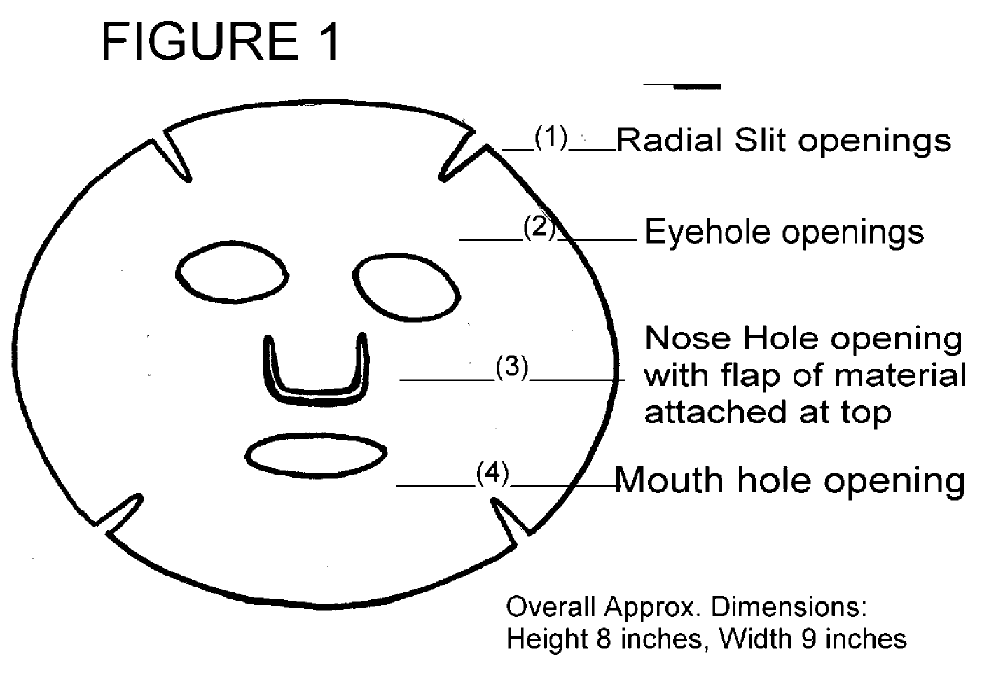

The preferred embodiment of the invention includes a face mask consisting of a piece of oval cotton material as shown in FIG. 1, with precut holes approximating the location of eyes and mouth and a precut “flap” in the nose area and precut radial lines to allow the oval to conform to the three dimensional contours of a human face. Such preferred embodiment of the face mask is shown in FIG. 1. However, it would be apparent to anyone skilled in the art, that the dimensions and shape of the face mask could be modified and variations could be made in the design of the precut openings, or that alternative materials, such as paper or polyester, woven or unwoven, or other natural or synthetic materials could be used in the fabrication of the face mask.

Also, as support to the face mask in its fabrication and to help retain the shape of the face mask in its packaging and in its removal, unfolding and handling by the user, an individual plastic liner may be used and packed, layered against the face mask in the same shape and size as the face mask itself. In its preferred embodiment, the face mask is folded in fourths (with the plastic liner folded with it), but it could also be folded in half, or other dimensions, in order to accommodate individual packaging.

The present invention allows a cosmetic, nutritional or medicinal preparation to remain in a liquid and absorbable state for a prolonged period of time while the mask is in place on the user's face. This allows for the continuous absorption by the skin of the liquid preparation impregnated in the mask. This continued absorption time of the topical preparation impregnated in the mask has several advantages in that it allows: (i) optimal time for the catalyst effect of various ingredients to interact with the skin; (ii) deeper absorption into the lower layers of the skin as the upper layers become and remain moist and saturated; (iii) longer effective treatment time as the preparation remains active while moist.

The face mask of the present invention would, in certain embodiments, be impregnated with a cosmetic preparation that would be beneficial to the facial skin, including, but not limited to herbal preparations, moisturizers, vitamins, nutritional supplements, amino acids, collagen, or other solutions for facial improvement.

Preferred cosmetic compositions of the present invention will contain from 30% to 100% water, preferably from 75% to 99.9%. Preferred cosmetic compositions may also contain polyhydric alcohols; preferably, but without limitation, propylene glycol.

Any aqueous based solution such as that presented in certain embodiments of the present invention requires some type of preservative to prevent the growth of potentially harmful microorganisms. In the present invention, a preservative that is non-irritating to the skin or eyes is necessary. The preferred preservative of the present invention is Germall BP, although many preservatives are known to one skilled in the art and could be substituted as appropriate in the present invention. The amount of preservative may range from 0.01% to 3%, with the preferred range of 0.1% to 0.5%.

Preferred cosmetic compositions of the present invention will contain natural botanical ingredients, including, but not limited to: Crocus of any species, preferably Crocus sativus, commonly called Tibet Red Flower/Saffron, or, Saussurea, of any species, commonly called Snow Lotus, Rhodiola of any species, cucumber, bamboo, licorice, hawthorn, algae, ginseng, wolfberry, sanchi root, aloe, collagen, amino acids, balsampear fruit also know as bitter gourd, olive or olive oil, white tea, green tea, black tea or grape extract. These and other botanical ingredients may include plant extracts, either water or butylene glycol based, purees of plant parts, or powdered plant parts. Amounts of botanical ingredients may range from 0.00001% to 10%, preferably from 0.001% to 3% by weight of the total composition.

Vitamins are also suitable for formulation in the composition of the present invention in combination with other active ingredients. Such anti-oxidants or vitamins include but are not limited to A, C, ascorbic acid, B's, such a B1, thiamine, B6, pyridoxine, B complex, biotin, choline, nicotinic acid, pantothenic acid, B12, cyanocobalamin, and /or B2, D, D2, D3, calciferol, E, such as tocopherol, riboflavin, K, K1, K2.

In other embodiments of the present invention, the face mask would be impregnated with a nutritional or medicinal solution which is to be topically delivered to the face for treatment of a skin condition or disease. The present inventions provides significantly increased effectiveness in the delivery of topical treatment in that the maintenance of a continuously wet application of solutions to the skin facilitates penetration through the five layers of the epidermis through to the underlying dermis.

Another embodiment of the present invention would have the face mask impregnated with an aromatic solution, as part of an aroma or therapy relaxation treatment. Fragrances could be of any composition, but would preferably be derived from natural botanical sources and essential oils, including without limitation, basil, bergamot, chamomile, cedarwood, citrus, clove, cypress, eucalyptus, fennel, frankincense, geranium, ginger, grapefruit, hyssop, jasmine, juniper, lavender, lemon, lemongrass, marjoram, myrrh, orange, peppermint, pine, rose, rosemary, saffron, sage, sandalwood, snow lotus, tea tree, thyme, ylang ylang. As with any aroma therapy, various scents would be incorporated for different therapeutic purposes, e.g. lavender for relaxation, peppermint for invigoration.

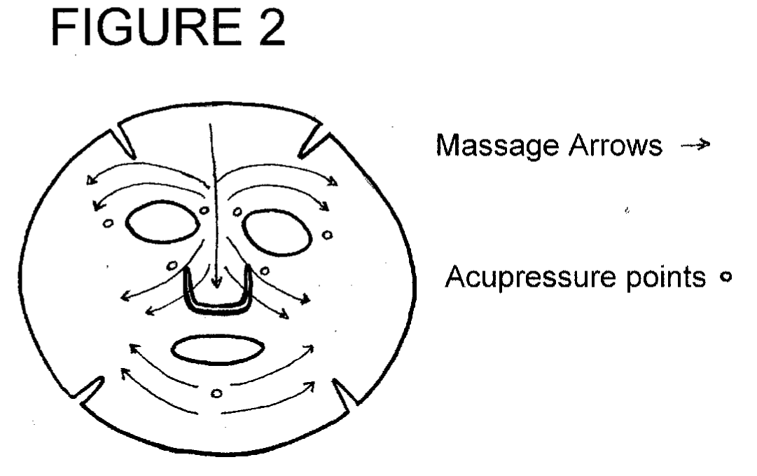

In combination with the aforementioned embodiments, or as a separate embodiment, the face mask would be printed on the side not placed against the skin with massage directional arrows and acupressure point locators to enhance and enable the face mask to be used as smooth, slippery surface for facial massage and acupressure point stimulation by the user or a third party. The face mask may be printed by any available technology, including thermal printing, electrophotography, inkjet, lithography, flexograpy, gravure, letterpress or screen. The preferred method is gravure, for its consistency and quality. Printed massage directions and acupressure points are displayed in FIG. 2 as illustrative of possible directions and points; however, it would be apparent to anyone skilled in the art that the printing could be modified to reflect particular massage techniques or acupressure points or directions for a particular branch of massage therapy or particular acupressure needs. Further, the directions could be color coded, so that, by way of example, acupressure points for headache relief would appear in red and acupressure points for relaxation would appear in blue and so on. Similarly, massage directions may vary, in that certain printings may be directed to different purposes or users, so that, by way of example, some masks may be printed with directions for Japanese facial massage and some may be printed with a “face-lifting” or anti-wrinkle directional arrows.

In the present invention, the face mask, whether preprinted or not, and the solution with which the face mask is impregnated or pre-moistened, are enclosed in a sealed container, preferably in individual laminated foil pouches, which can then be packed in boxes containing several pouches, preferably 8 to 12, or alternatively in a resealable jar or plastic container containing several face masks separated by their plastic liners. The advantage of the individual pouches is that each mask remains moist and fresh, ready for use. Futhermore, in the case of medicinal solutions, prescribed quantities of active ingredients can be measured and controlled for each application of a mask.

Application of natural herb products and vitamins can be beneficial to the general health as well as skin and may be considered to be a “natural skin supplement” offering the health advantages of natural vitamins and amino acids in an aromatic, soothing, enjoyable, and healthful skin application.

Other active agents which can be used with the present invention include e.g. antioxidants, anti-carcinogens, anti-inflammatory agents, hormones, antibiotics, collagen, anti-bacterial agents and other medically useful drugs, supplements or herbs such as those identified in current medical literature, such as those contained in: 2004 Physicians's Desk Reference, Thomson Healthcare Publishing Company; 58^{th} Bk&Cr edition (Nov. 1, 2003).

The various preferred compositions and active agents and ingredients described herein are illustrative only and could include others, such as those known and identified in current literature such as: PDR for Herbal Medicines, Third Edition, Thomson Healthcare Publishing Company (Dec. 30, 2004); and PDR for Nutritional Supplements, First Edition, Thomson Healthcare Publishing Company (Mar. 15, 2001).

No statement of “preferred embodiment” or “preferred composition” is to be read to exclude any other possible embodiment included in the Claims or that can be derived from the Claims, all described embodiments are to be considered in all respects only as illustrative, and not restrictive. All of the embodiments and/or methods disclosed and claimed herein can be made and executed without undue experimentation in light of the present disclosure. While the embodiments and methods of this invention have been described in terms of preferred embodiments, it will be apparent to those of skill in the art that variations may be applied to the embodiments and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended Claims.

What is claimed is:

1. A cosmetic product comprising:
(i) a sealed container,
(ii) an absorbent material, in an oval shape with openings corresponding in approximate size and position to human eyes, nose and mouth and radial cuts to allow the shaping of the flat material around the three dimensional contours of a face, such material being enclosed in the sealed container,
(iii) a cosmetic composition in the sealed container which is at least partially absorbed into and on the surface of the absorbent material.

2. The product according to claim 1 wherein the material is 20% to 100% woven cotton.

3. The product according to claim 1 wherein the material is 20% to 100% non woven polyester substrate.

4. The product according to claim 1 wherein the material is 20% to 100% paper or other wood derived product.

5. The product according to claim 1 wherein the material is lined with a disposable plastic liner.

6. The product according to claim 1 wherein the material is printed with massage directions.

7. The product according to claim 1 wherein the material is printed with the location of facial acupressure points.

8. The product according to claim 1 wherein the cosmetic composition contains water ranges from 1% to 99.9%.

9. The product according to claim 1 wherein the cosmetic composition contains herbal components.

10. The product according to claim 1 wherein the cosmetic composition contains skin nutrients.

11. The product according to claim 1 wherein the cosmetic composition contains collagen.

12. The product according to claim 1 wherein the cosmetic composition contains skin moisturizer.

13. The product according to claim 1 wherein the cosmetic composition contains vitamins, amino acids or nutritional supplements.

14. The product according to claim 1 wherein the cosmetic composition contains aromatic components.

15. The product according to claim 1 wherein the cosmetic composition contains medicinal components.

16. A method for applying cosmetic compositions to the face to treat the conditions of skin aging, wrinkles, mottled or uneven skin tone and color, dull or dry skin, redness, acne, rosecea, eczema oiliness, blemishes, or combinations thereof, comprising application over the face of a material, with holes for eyes, nose and mouth, impregnated with a cosmetic composition.

17. The method according claim 16 wherein the material is impregnated with a medicinal component.

18. The method according to claim 16 wherein the material is impregnated with an aromatic component.

19. The method according to claim 16 wherein the material, pre-moistened with an aqueous solution, provides a smooth, slippery surface to be used for facial massage and/or facial acupressure point pressure.

20. The method according to claim 16 wherein the material is printed with massage directions or acupressure point locations.

21. A facial mask made of an absorbent material, in an oval shape with openings corresponding in approximate size and position to human eyes, nose and mouth and radial cuts to allow the shaping of the flat material around the three dimensional contours of a face.

22. The facial mask according to claim 21 wherein the absorbent material is printed with massage directions, acupressure point locations or trademark, corporate or decorative designs.
